(19) United States
(12) Patent Application Publication
(10) Pub. No.: US 2022/0249020 A1
(43) Pub. Date: Aug. 11, 2022

(54) SYSTEM FOR MONITORING PHYSIOLOGICAL PARAMETERS

(71) Applicant: Evonik Operations GmbH, Essen (DE)

(72) Inventors: Rosario LIZIO, Muenster (DE); Sara LIEBANA VIÑAS, Aschaffenburg (DE); Philipp OCKERMANN, Wuerzburg (DE); Jean-Luc HERBEAUX, Cascais (PT)

(73) Assignee: Evonik Operations GmbH, Essen (DE)

(21) Appl. No.: 17/614,138

(22) PCT Filed: May 26, 2020
(86) PCT No.: PCT/EP2020/064537

§ 371 Date: Nov. 24, 2021

(30) Foreign Application Priority Data
May 27, 2019 (EP) 19176667.4

(51) Int. Cl. A61B 5/00; A61B 5/1455; A61B 5/352; A61B 5/0205; A61B 5/021; A61B 5/024; G16H 20/60; G16H 50/30; A61B 5/11

(52) U.S. Cl. CPC A61B 5/4836; A61B 5/14551; A61B 5/352; A61B 5/0205; A61B 5/02108; A61B 5/02405; A61B 2562/0219; A61B 5/4866; A61B 5/4869; A61B 5/7465; G16H 20/60; G16H 50/30; A61B 5/1112; A61B 5/02416

(57) ABSTRACT

The present invention relates to a system for monitoring physiological parameters to an integrated digital system, which is able to determine several biological parameters, such as from photoplethysmographic (PPG) signals and other connected devices or sensors to give a personalized supplement, nutritional and lifestyle recommendation to improve specifically said parameters. By using new algorithms based on PPG signals the cardiovascular condition of a person can be analyzed by estimating cardiovascular parameters.

The present invention relates to a system for monitoring physiological parameters to an integrated digital system, which is able to determine several biological parameters, such as from photoplethysmographic (PPG) signals and other connected devices or sensors to give a personalized supplement, nutritional and lifestyle recommendation to improve specifically said parameters.

Several digital systems have been described in the literature providing nutritional recommendations in connection with the measurement of physiological parameters of a user.

US2017/0148348A1 for instance described a digital system aiming to give a personalized vitamin supplement recommendation starting from the measurement of physiological and/or environmental factors estimating a general nutritional deficiency and give a suggestion how to overcome such deficiency. That system however is not able to visualize and show the improvement of the specific biological functions after supplementation in a normal case, where no pathological deficiencies have been determined.

US2014/221784A1 describes a system capable to collect sensor data to derived physical and psychological “health-related characteristic” of the user, who has to express his own assessment (target) allowing thereof to the system to suggest a nutrition modification, mainly in the field of calories intake and consumption. Also, there is not an automated correlation between measured parameters and specific nutritional suggestion for the improvement of the specific parameter.

US 2014/0127650A1 discloses an apparatus and management method to ensure general health and wellness starting from subjective users data to generate a nutrition profile and comparing that profile (nutritional score) with reference data to determine a nutrition deficiency. The final nutritional suggestion aims to compensate that deficiency within the general categories of carbohydrates, lipids, proteins and water under consideration of the specific energy consumptions as measured from the activity level of the user. Even in this case there is not a direct correlation between measured parameters and specific nutritional suggestions to improve said parameter.

Similarly, in CN103984847A a system is described that uses physiological parameters to determine the “Physical Condition” of the user and generate a food and drink recommendation for the corresponding category of user.

However, none of the available system is able to give specific supplement, nutritional and lifestyle recommendations to improve the measured physiological parameter of the individual user and to visualize and monitor the related improvements.

Therefore, proceeding from the prior art, there is a need for a health monitoring system, which can provide specific personal suggestions for food and advanced food ingredients based on the evaluation of physiological parameters of the user, which are calculated on the basis of measured signals obtained from various sensors, such as PPG sensors, which may be integrated in a fitness tracker or a smartwatch.

The aim of the invention is to monitor, visualize and maintain the biological parameter as close as possible to the ideal value due to one or more supplements and other lifestyle connected suggestions in order to prevent illness and improve or maintain the wellbeing and healthy status of the user.

The problem is solved by providing a system for monitoring physiological parameters of a user comprising:
A human body health monitoring device comprising at least one sensor adapted to obtain primary physiological signals of the user;
A processing system communicatively coupled to the sensor adapted to
   calculate one or more physiological parameters based on the primary physiological signals and based on individual parameters of the user,
   compare the calculated physiological parameters with prestored physiological index parameters, and determine a specific deviation between the calculated physiological parameters to the prestored physiological index parameters,
   compare the specific deviation(s) with a database containing nutrients, nutraceuticals, advanced food ingredients and single nutritional components specifically selected via scientific and clinical studies to have a specific positive/normalizing effect on said physiological parameters,
   provide a nutritional suggestion to the user for the normalization of the physiological parameters based on the comparison of the specific deviation(s) with the nutritional database; and
Output means adapted to output the calculated physiological parameters and the nutritional suggestion.

In a preferred embodiment, the physiological parameters calculated are cardiovascular health parameters, cognitive health parameters, gut health parameters, metabolic parameters, body mass and body efficiency parameters, stress and sleep parameters or inflammatory parameters, metabolic dysfunctions or a combination thereof.

In a specific embodiment, the physiological parameters calculated are cardiovascular health parameters chosen from vascular age index AgIxPPG (parameter that gives information on the age condition of the arteries, compared to some normal threshold for a healthy population), blood pressure BPdia and BPsys (pressure that the blood traveling through a large artery exerts onto its walls), pulse wave velocity PWV (describing the velocity of blood that travels through a person's arteries and being defined as the speed at which the pressure wave propagates through the cardiovascular tree), augmentation index AIxPPG (indirect measure of arterial stiffness, which provides information about the pressure wave reflection by the peripheral circulatory system) and heart rate variability HRV.

The HRV is the fluctuation in the time intervals between adjacent heartbeats and is preferably calculated in form of Root Mean Square of Successive Differences (RMSSD) between normal heartbeats. The RMSSD reflects the beat-to-beat variance in HR and is the primary time-domain measure used to estimate the vagally mediated changes reflected in HRV. The RMSSD is obtained by first calculating each successive time difference between heartbeats in ms. Then, each of the values is squared and the result is averaged before the square root of the total is obtained. The conventional minimum recording is 5 min (Shaffer and Ginsberg, Frontiers in Public Health Vol. 5, Art. 258, September 2017).

The RMSSD is calculated with the following formula:

$$RMSSD = \sqrt{\frac{1}{N - 1} ( {\sum }_{i = 1}^{N - 1} {( {( RR )}_{i + 1} - {( RR )}_{i} )}^{2}}$$

RR: RR interval, time difference of succeeding R peaks in the ECG

N: number of R peaks in the ECG

The sensor according to the present invention is chosen from one or more of the following:
Photoplethysmographic (PPG) sensor
Bioimpedance sensor
Pulse Oximeter
Capacitive sensor
Temperature sensor
Humidity sensor
Ultraviolet (UV) sensor
Ambient light sensor
3 (or more) axis accelerometer
Altimeter
Barometer
Compass
Gyroscope
Magnetometer
Gesture technology
Global Positioning System (GPS)
Long Term Evolution (LTE).

In an advantageous configuration of the present invention, the sensor is chosen from one ore more of the following:
Photoplethysmographic (PPG) sensor
Bioimpedance sensor
3 axis accelerometer
Altimeter
Barometer
Gyroscope
Global Positioning System (GPS)

In a further preferred embodiment, the sensor is a PPG sensor, which can be found in a number of different devices. Not only are they built into consumer goods such as wrist-type fitness trackers but also into devices used by medical professionals. The sensors are mostly used to either estimate the pulse rate or the oxygen saturation in the blood. It is further preferred to use two or more PPG sensors.

In a specific embodiment, the system comprises two PPG sensors and the system further comprises a bioimpedance sensor. The bioimpedance sensor can allow continuous surveillance of blood glucose level and is relevant in pre-diabetic health assessment. Taking into consideration the blood glucose level of the user, specific nutritional recommendations can be given.

A plethysmograph is an instrument that measures changes in volume of an organ and is basically an optical sensor. The term photoplethysmography usually refers to the measurement of volume changes in arteries and arterioles due to blood flow. There are different kinds of PPG sensors. Some are placed at the fingertip, some at the wrist and other sites such as the ear lobe are also possible. The sensor itself consists of a light emitting diode (LED) that emits light onto the skin and of a photodiode. This diode is usually placed next to the LED, detecting light that is reflected (Type B). For finger sensors, the photodiode can also be placed at the opposite end of the finger, measuring the light that travels through the finger (Type A).

The calculation of one or more physiological parameters based on the primary physiological signals, such as PPG signals from a wearable device or other connected sensors and on individual parameters of the user is achieved with the help of advanced algorithms, considering various parameters, such as the age, the height or the heart rate of the user. By incorporation of specific anatomical data of the user, the algorithms provide a more precise estimation of the physiological parameters.

Therefore, in an advantageous configuration of the present invention, one or more physiological parameters are calculated based on the primary physiological signals using linear regression on parameters, selected from age, height and the heart rate of the user.

With such algorithms, further cardiovascular parameters can be extracted from PPG signals, which are not analyzed in conventional fitness tracker, such as augmentation index, vessel elasticity, pulse wave velocity and blood pressure. Normally, PPG is used to determine pulse rate and oxygen saturation. These supplementary parameters are beneficial for a comprehensive general health assessment and lead to reduction of the risk of misinterpretation of physiological parameters and allow new health predictions. Thereby, an individual and more precise cardiovascular health status assessment can be achieved.

In a preferred configuration, the following parameters related to cardiovascular (CV) health are calculated based on the measured PPG signals of two or more PPG sensors arranged in a distinct distance.

In a preferred configuration, two PPG sensors are used and positioned with a distance of 5 cm or less between the two PPG sensors, preferably between 1 cm and 4 cm. It is possible to include the two sensors in two distinct wrist-worn devices or into one wrist-worn device. Alternatively, one PPG sensor is located in a wrist-worn device and another PPG sensor is located into another device, such as a ring ora health monitoring device, which is included within clothing or shoes of the user. However, it is preferred to include two PPG sensors within one wrist-worn device.

In a preferred configuration, the system is configured to determine one or more cardiovascular parameters in a user, the user having an age and a body height with the following steps:
determining the age (p_{age}) and body height (p_{height}) of the user,
measuring at least two photoplethysmographic (PPG) signals with at least two PPG sensors at two different positions at the user,
separating the PPG signal into PPG pulses, whereby the start point and the end point of the pulse corresponds the systolic foot of the PPG signal,
determining the heart rate of the subject (p_{HR}) and calculating the median heart rate,
determining the systolic A_{sys} and diastolic A_{dia} peak amplitudes and their times is and t_{d},
calculating the second derivative of the PPG pulse, and determining the characteristic points a, b, c, d, and e from the second derivative of the PPG pulse, wherein

a and e are the first and second most prominent maxima in the second derivative, respectively,

c is the most prominent peak between the points a and e,

b is the most prominent minimum in the second derivative and,

d is the most prominent minimum between points c and e,
determining:
   a) the vascular age index AgIx using linear regression based on the characteristic points a, b, c, d, and e, age (p_{age}), body height (p) and median heart rate of the user,
   b) the pulse wave velocity PVW using linear regression based on the time difference between the two PPG pulses (PTT), age (p_{age}), body height (p_{height}) and median heart rate estimation of the user,
   c) blood pressure BP_{dia} and BP_{sys} using linear regression based on time difference between the two PPG pulses (PTT) and median heart rate and
   d) optionally the augmentation index AIx, based on the systolic A_{sys} and diastolic A_{dia} peak amplitudes normalized to 75 heartbeats (AIx@75) and using a linear regression based on the normalized augmentation index AIx.

A plethysmographic (PPG) measurement can provide several parameters and indicators, thanks to which it's possible to obtain information about the cardiovascular system. The continuous research for new parameters is driven by the high portability of a photopletysmographic system: the classical measurement technique, which often involves bulky instrument, could be replaced with this kind of instrument, that is easy to set up and also allows continuous monitoring.

Elgendi (Current Cardiology Reviews, 2012, 8, 14-25) describes the use of PPG to estimate the skin blood flow using infrared light. Recent studies emphasize the potential information embedded in the PPG waveform signal and it deserves further attention for its possible applications beyond pulse oximetry and heart-rate calculation. Especially, characteristics of the PPG waveform and its derivatives may serve as a basis for evaluating vascular stiffness and aging indices.

Separation of PPG Signal into Pulses

In order to analyse each individual PPG waveform in the PPG signal and to reduce the effect of motion artefacts, the PPG signal is not examined as a whole but in sections. According to the present invention the signal is divided into individual pulses, as all features which are extracted from the PPG signal can be derived from one pulse wave. The systolic foot is the most prominent feature of a PPG pulse and can therefore be found most reliably in the PPG signal. Therefore, the PPG signal was chopped into PPG pulses at this systolic foot by finding the minima in the PPG signal. This strategy allows to analyse each pulse individually. If a few pulses are not correctly recognized, this does not have a falsifying effect on the final results for a measurement as the final parameter values are calculated by the median of all individual pulses' results.

Other PPG Parameters

Various morphological characteristics of the PPG signal and its derivatives have also been studied:

The Pulse Area is defined as the area under the PPG curve. In a recent study (Usman et al., Acta Scientiarum Technology, vol. 36, n. 1, pp. 123-128, 2013), a significant difference in this parameter was found in relation to two different levels of diabetes. In conclusion, the authors affirmed that it can be used as a useful parameter in determining arterial stiffness. In the work of Wang et al. (Annual International Conferente of the IEEE Engineering in Medicine and Biology Society, 2009), the area is divided into two sub-areas, A1 and A2, at the dicrotic notch. Based on these two measures, the Inflection Point Ratio was defined as the ratio between the two areas, demonstrating that this ratio can be used as an indicator of total peripheral resistance.

The time ΔT between the systolic peak and the diastolic peak seems to be linked to the blood vessels elasticity. Millasseau et al. (Clinical Science, vol. 103, n. 4, pp. 371-377, 2002) used this time interval to obtain a new index, the Large Artery Stiffness Index (SI), defined as the ratio between the height of the subject and the time interval between the systolic and diastolic peaks, finding that it decreases with age.

Another measure of the PPG signal temporal trend is the Crest Time (CT). Easy to measure, the CT is the time elapsed between the systolic foot and the systolic peak of a PPG wave. It has been assessed as a valid parameter (together with other measurements deriving from the PPG signal) for a cheap and effective Cardiovascular Disease (CVD) screening technique for use in general clinical practice (Alty et al., IEEE Transactions on biomedical engineering, vol. 54, n. 12, pp. 2268-2275, 2007).

The CT and the SI can be estimated in a more reliable way using the first derivative of the PPG signal, also known as Velocity Photoplethysmograph (VPG), measuring the time interval between the relative zero-cross.

Parameter Estimates

1. Augmentation Index (AIx_{PPG}):

An indirect measure of arterial stiffness can be provided by the Augmentation Index (AIx). It provides information about the pressure wave reflection by the peripheral circulatory system. The Augmentation Index measure was transposed from the Blood Pressure Pulse Wave Analysis to the PPG signal, assuming that one is able to obtain information about the arterial stiffness analyzing the PPG waveform.

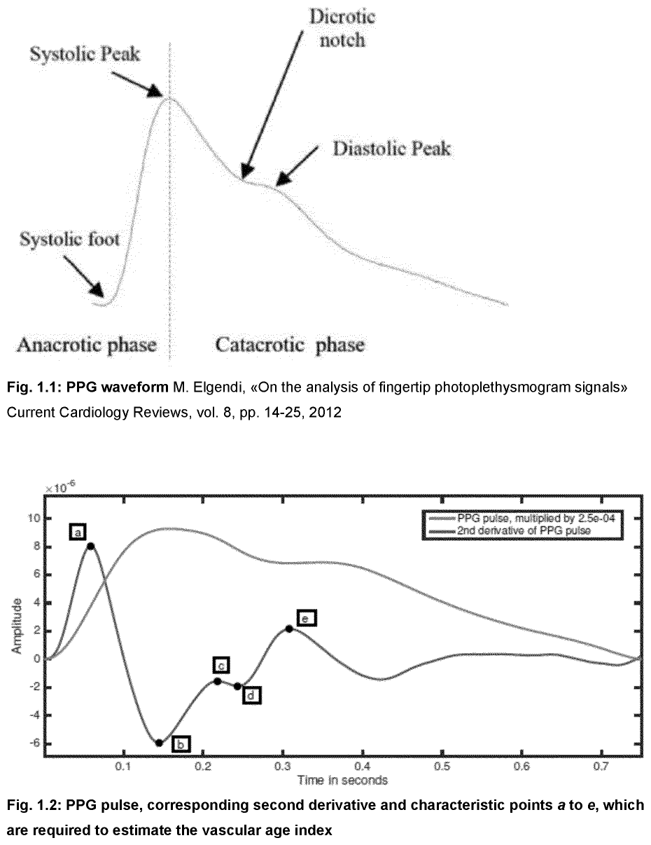

The PPG pulse wave is not a pressure pulse wave. Thus, the augmentation index as described above be obtained directly from the PPG signal. Generally, the Augmentation Index can be estimated thanks to the PPG morphological properties. According to literature, the augmentation index is calculated with the help of the following formula:

$$\begin{matrix}AIx = \frac{y}{x} & ( 1.1 ) \\ AIx = \frac{x - y}{x} & ( 1.2 )\end{matrix}$$

wherein y is the diastolic peak amplitude and x is the systolic peak amplitude (as shown in FIG. 1.1).

The AIx describes the augmentation of the PPG signal from the systolic to the diastolic peak.

From the PPG pulse wave, the systolic A_{sys} and diastolic A_{dia} peak amplitudes are estimated (corresponding to x and y in formula 1.2 respectively), as well as their times tₛ and t_{d}. The determination of A_{dia} in the PPG waveform can be very difficult when the reflected wave is very small and there is no visible diastolic peak in the waveform (see FIG. 1.1). To still be able to estimate both peak positions, two different methods to model the form of the two waves were developed.

In the first method, the PPG waveform is modelled as a sum of the two pulse waves through exponential functions.

$$\begin{matrix}{y}_{pulse} ( t ) = {y}_{sys} ( t ) + {y}_{dia} ( t ) \\ = {b}_{1} {e}^{\frac{- {( t - {\hat{t}}_{s} )}^{2}}{{b}_{2}}} + {b}_{3} {e}^{\frac{- {( t - {\hat{t}}_{d} )}^{2}}{{b}_{4}}}\end{matrix} \qquad ( 1.3 )$$

Nonlinear regression is applied to fit the model to the PPG waveform and receive estimates of is and t_{d} to find A_{sys} and A_{dia}, respectively.

The second method makes use of the fact that the maximum in the PPG waveform is the systolic peak. By modelling only the first wave with known position at the systolic peak, its exponential model is substracted from the PPG signal and yield the remaining reflected wave,

$$\begin{matrix}{y}_{dia} ( t ) = {y}_{pulse} ( t ) + {y}_{sys} ( t ) \\ = {y}_{pulse} ( t ) - {b}_{1} {e}^{\frac{- {( t - {\hat{t}}_{s} )}^{2}}{{b}_{2}}}\end{matrix} \qquad ( 1.4 )$$

whose maximal value max y_{dia}(t)=A_{dia} and and \hat{t}_{d} is the corresponding diastolic time index estimate.

A parameter that seems to be more reliable is the Augmentation Index normalized to 75 heartbeats (AIx@75). Indeed, it seems that this parameter depends on the heartbeat. It was introduced for the first time in the work of Wilkinson et al. (American Journal of Hypertension, vol. 15, pp. 24-30, 2002). It has been found that the AIx estimated from the Blood Pressure wave has different values compared to the same parameter estimated from the PPG wave. Thus, the AIx and the AIx@75 were used in a linear regression with the reference values. Same methods were applied to calculate both the AIx and AIx@75.

The normalized index value AIx@75 was obtained and in used in linear regression model:

AIx@75=b₀+b₁A [symbol] ; (1.5)

Feature Extraction from Signal's Derivatives

Other features are obtained from the signal's derivatives which are calculated by the differences between adjacent samples. A moving average filter was applied to remove high frequency noise introduced by taking the derivative. To reliably find the characteristic points a to e, an algorithm to find the two most prominent maxima was developed and they were marked as a and e, respectively. The point c is then the most prominent peak between point a and e. Furthermore, point b is the most prominent minimum in the second derivative and point d is the most prominent minimum between points c and e (see FIG. 1.2).

Therefore, in a preferred embodiment of the present invention the characteristic points a, b, c, d, and e are automatically derived from the second derivative of the PPG pulse, wherein a and e are the first and second most prominent maxima in the second derivative, respectively, c is the most prominent peak between the points a and e, b is the most prominent minimum in the second derivative and, d is the most prominent minimum between points c and e.

2. Vascular Age Index (AgIx_{PPG}):

Regarding the PPG waveform, a Vascular Age Index estimate can be obtained through the analysis of the second derivative of the PPG signal, also known as Acceleration Photoplethysmography (APG). It is characterized by several landmark points, like the PPG wave; the estimation of these points is used to obtain indicators that give information about the cardiovascular function, including the Vascular Age Index. The state-of-the-art literature calculates a ratio of the characteristic points by

$$= 45.5 * \frac{b - c - d - e}{a} + 65.9 \qquad ( 1.6 )$$

The index describes the cardiovascular age of a person. It should be lower than the person's chronological age if their vessels aged slower than average and higher than their chronological age otherwise.

Despite the most used parameter from the APG is the Vascular Age Index, other measures have been investigated starting from the APG wave estimates, for example, ratios between the b, c, d or e wave and a wave in several studies (Elgendi, Current Cardiology Reviews, vol. 8, pp. 14-25, 2012). It has been found that these ratios vary with the subject age. As a Vascular Age Index alternative, in case of the c and d waves are not visible, the (b−e)/a ratio could be used, as suggested in another study (Baek et al., 6th International Special Topic Conference on Information Technology Applications in Biomedicine, 2007).

In addition to the Vascular Age Index, this index was also estimated:

$$\frac{b - e}{a} \qquad ( 1.7 )$$

To more reliable estimate AgIx, a new linear regression model with coefficients dᵢ based on the estimated Vascular Age Index [symbol] , which is based on the characteristic points a, b, c, d and e was developed:

AgIx=d₀+d₁ [symbol] +d₂p_{age}+d₃p_{height}+d₄ [symbol] (1.8)

wherein dᵢ are the coefficients, p_{age} is the age, p_{height} is the height, [symbol] is the median heart rate estimate of a person.

3. Pulse Wave Velocity (PWV):

The PWV is measured experimentally as the ratio between the distance between two different measurement sites on the same line through which the pressure wave propagates, and the time interval between wave corresponding points.

The Pulse Wave Velocity can be estimated also with the PPG signal. In this case, the PWV can be obtained with two different instrumental setups:
ECG+PPG sensor: one has to evaluate the Pulse Arrival Time (PAT) as the time interval between the ECG R peak and a PPG landmark point (systolic foot, max gradient or systolic peak);
2 PPG sensors: they are positioned one downstream of the other and, in this case, one has to evaluate the Pulse Transit Time (PTT) as the time interval between the two measurement sites.

It is necessary to distinguish and specify the measured time interval: the PAT is equal to the sum of PTT and the Pre-Ejection Period (PEP), that is the time interval between the beginning of the ventricular depolarization and the moment in which the aortic valve opens. Since PEP is difficult to measure or predict and is not a linear function of pressure, it turns out that PAT is a less accurate indicator than the PTT. Although it is more difficult to assess, PTT provides a better measure for monitoring. This parameter would allow estimating the aortic PWV (the aorta is the reference point to measure the PWV in the literature). Modern pressure measurement systems also calculate aortic PWV with indirect methods.

To obtain a PWV estimate, PPG signals systolic feet from two different measurement systems are identified. Thanks to the difference between the time instants at which the systolic feet are recorded, it is possible to know the Pulse Arrival Time and the Pulse Transit Time, depending on the instruments (ECG and PPG in the first case, two PPG signals in the second). This measure will be used to evaluate the correlation between the PAT or the PTT and the Pulse Wave Velocity measured from the gold standard instrument, which refers to the central PWV, i.e. in the aorta. For this reason, a linear regression was created using Pulse Transit Time values, age, height, median heart rate value and three typical parameters of the PPG signal, i.e. Crest Time, Stiffness Index and Pulse Area.

The PWV is estimated by the time difference between pulses of two PPG signals measured at two separately placed PPG sensors (here the PTT). Therefore, the time difference between the systolic feet of the signals is examined. The median time differences are used for a linear regression model to estimate the PWV. Additional physiological and personal data were further included in the linear regression model:

PWV=g₀+g₁ [symbol] +g₂p_{age}+g₃p_{height}+g₄ [symbol] (1.9)

wherein gᵢ are the coefficients, PTT is the time difference between the PPG pulses, p_{age} is the age, p_{height} is the height and [symbol] is the median heart rate of a person.

It is preferred that two PPG signals are measured and the time difference between the two corresponding PPG pulses are considered. In one embodiment, one PPG sensor can be positioned at the wrist of a user and the second sensor can be positioned at the finger of a user. However, in an advantageous configuration, two PPG sensors can be positioned at the wrist of a user with a certain distance between both sensors. This allows the implementation in wrist-worn devices, such as smartwatches or fitness trackers.

4. Blood Pressure (BP):

The blood pressure estimate from the PPG signal is not such a trivial task. Previous studies suggest to estimate the BP by a simple linear regression model using the extracted systolic and diastolic times of a PPG pulse:

BP_{dia}=a_{SBP}t_{dia}+b_{SBP} (1.10)

BP_{sys}=a_{DBP}t_{sys}+b_{DBP} (1.11)

Wherein a_{SBP}, b_{SBP}, a_{DBP} and b_{DBP} are coefficients that have to be estimated based on reference values.

For the present invention a strategy for estimating the arterial blood pressure (systolic and diastolic) was developed, working on the Pulse Transit Time and evaluating the linear regression of these values with the blood pressure estimates obtained with the gold standard instrument. Furthermore, other parameters were used in the linear regression estimates, like the median heart rate, Crest Time, Stiffness Index and Pulse Area and physiological parameters, such as age and height.

BP_{sys}=k₀ₛ+k₁ₛ [symbol] +k₂ₛp_{age}+k₃ₛp_{height}+k₄ₛ [symbol] (1.12)

BP_{dia}=k_{0d}+k_{1d} [symbol] +k_{2d}p_{age}+k_{3d}p_{height}+k_{4d} [symbol] (1.13)

BP_{sys}=l₀ₛ+l₁ₛ [symbol] +l₂ₛ [symbol] +l₃ₛCTₚ+l₄ₛSIₚ+l₅ₛPAₚ (1.14)

BP_{dia}=l_{0d}+l_{1d} [symbol] +l_{2d} [symbol] +l_{3d}CTₚ+l_{4d}SIₚ+l_{5d}PAₚ (1.15)

wherein k₀ₛ to k₂ₛ, k_{0d} to k_{2d}, l_{0d} to l_{5d}, l₀ₛ to l₅ₛ, are the coefficients, [symbol] is the time difference between the PPG pulses, p_{age} is the age, p_{height} is the height and [symbol] is the median heart rate of a person, CTₚ is the Crest Time, SIₚ is Stiffness Index and PAₚ is the Pulse Area of the PPG signal from the proximal sensor.

5. Heart Rate Variability (HRV):

The heart rate variability (HRV) describes the variation in the time interval between heartbeats. The interbeat interval (IBI) value for each heartbeat is estimated as the time interval between two corresponding landmark points of two consecutive PPG waves (systolic foot, max gradient or systolic peak). In a preferred configuration, the IBI is measured as the time interval between two consecutive systolic feet.

Once the IBIs have been measured, it is possible to estimate the HRV parameters. Conventionally, HRV analysis is performed in the time domain and in the frequency domain. In addition, some of these parameters can only be estimated if the recording has a sufficiently long duration. For short recordings (i.e. two minutes at least), the following are some of the possible indices that can be obtained (Shaffer and Ginsberg, Frontiers in Public Health, vol. 5, n. 258, p. 17 pp, 2017):
1. Standard Deviation of the IBI of normal sinus beats (SDNN)
2. Number of adjacent intervals that differ from each other by more than 50 ms (NN50 and pNN50)
3. Root Mean Square of Successive Difference between normal heartbeats (RMSSD), obtained by first calculating each successive time difference between heartbeats; then, each of the values is squared and the result is averaged before the square root of the total
4. LF/HF ratio, the ratio between the low-frequency power (0.04-0.15 Hz) and the high-frequency power (0.15-0.4 Hz)
5. Poincare Plot, it is obtained by plotting every IBI interval against the prior interval, creating a scatter plot; the Poincare Plot can also be analyzed by fitting an ellipse to the plotted points. After the fitting phase, two non-linear measurements can be obtained:
   5.a. SD1: standard deviation of the distance of each point from the x-axis, specifies the ellipse's width; it reflects short-term HRV
   5.b. SD2: standard deviation of each point from the y=x+mean(IBI interval), it specifies the ellipse's length; it measures the short- and long-term HRV
6. Sample Entropy, which measures the regularity and complexity of the time series.

An increasing number of wearable devices claim to provide accurate, economic and easily measurable HRV indices using PPG technique. Several studies have focused on the reliability of the HRV indices reported by PPG measurements compared to the gold standard, given by the ECG signal. In particular, in a recent review (Georgiou et al., Folia Medica, vol. 60, n. 1, pp. 7-20, 2018) the result that emerges is that PPG technology can be a valid alternative for HRV measurements, although it is still necessary to conduct more in-depth studies under non-stationary conditions.

In a preferred configuration, the method further comprises the determination of Crest Time (CT), Stiffness Index (SI) and Pulse Area (PA) of the PPG signal and wherein the cardiovascular parameters are estimated with the following equations:
a) vascular age index AgIx:
   AgIx=d₀+d₁ [symbol] +d₂p_{age}+d₃p_{height}+d₄ [symbol] , wherein [symbol] is estimated based on characteristic points a, b, c, d, and e:

$$= 45.4 * \frac{b - c - d - e}{a} + 65.9 ;$$

b) pulse wave velocity PWV:

PWV=g₀+g₁ [symbol] +g₂p_{age}+g₃p_{height}+g₄ [symbol] ;
c) blood pressure BP_{dia} and BP_{sys}:

BP_{dia}=l_{0d}+l_{1d} [symbol] +l_{2d} [symbol] +l_{3d}CTₚ+l_{4d}SIₚ+l_{5d}PAₚ

BP_{sys}=k₀ₛ+k₁ₛ [symbol] +k₂ₛ [symbol] ;
d) normalized augmentation index AIx@75:
   [symbol] =(x−y)/y by the sum of two exponential, and
   AIx@75=b₀+b₁ [symbol] , wherein AIx@75 is the augmentation index (AIx) normalized to 75 heartbeats;
wherein, p_{age} is the age and p_{height} is the body height of the subject, median (HR) is the median heart rate, PTT is the time difference between the PPG pulses, A_{sys} and A_{dia} are magnitudes of the systolic and diastolic peak, respectively, CT is the Crest Time, ST is the Stiffness Index and PA is the Pulse Area of the PPG signal, d₀ to d₄, g₀ to g₄, l_{0d} to l_{kd}, k₀ₛ to k₂ₛ, and b₀ to b₁ represent the coefficients of the respective linear regression equation.

In a preferred configuration, the cardiovascular parameters are estimated based on at least 60 PPG pulses, preferably at least 100 PPG pulses, more preferably at least 120 PPG pulses. The estimation of 60 pulses corresponds to measurement time of approximately 1 minute (with 60 pulses per minute). Therefore, the preferred configurations refer to a measurement time of at least 1 minute (60 PPG pulses), preferably at least 1.7 minutes (100 PPG pulses), more preferably at least 2 minutes (120 PPG pulses). By combining the results obtained by every PPG pulse mediated in the measured time, this allows a more reliable estimation. In this way, if there is a corrupted PPG pulse, its effect can be smoothed if the signals are mediated over the measured time. The measurement of PPG pulses over a defined time has the advantage that the single PPG pulses do not need to be classified as it necessary in the state of the art (e.g. such as in US2013/324859A1) and this provides a more efficient algorithm.

In alternative configurations, additionally to one, two, three or four cardiovascular parameters, the heart rate variability HRV is determined by calculating one or more of the following
Minimum and maximum interbeat interval (IBI)
Median and mean IBI
Minimum and maximum heart rate
Median and mean heart rate
Standard Deviation of the IBI of normal sinus beats (SDNN)
Number of adjacent intervals that differ from each other by more than 50 ms (NN50 and pNN50)
Root Mean Square of Successive Difference between normal heartbeats (RMSSD),
LF/HF ratio, the ratio between the low-frequency power (0.04-0.15 Hz) and the high-frequency power (0.15-0.4 Hz)
SD1: standard deviation of the distance of each point from the x-axis in a Poincare Plot, obtained by plotting every IBI interval against the prior interval
SD2: standard deviation of each point from the y=x+mean (IBI interval) in a Poincare Plot, obtained by plotting every IBI interval against the prior interval
Sample Entropy.

According to the present invention, primary physiological parameters are determined. Moreover, secondary physiological parameters may also be determined, which can be a derived from a combination of several primary physiological parameters, or a combination with metadata from the user (such as age, height, weight).

By determining secondary physiological parameters such as blood flow, blood pressure, arterial stiffness/vessel elasticity or vascular age a more comprehensive general health assessment can be provided. Moreover, new secondary parameters based on primary physiological parameters and/or metadata of the user, such as stress level, fitness index, recovery index, cardiovascular index or biological age can be determined. The analysis of these supplemental parameters leads to a reduction of misinterpretation risk and allows an individual CV-health status assessment. The measurement of new parameters allows new holistic health monitoring and more precise health predictions.

The calculated physiological parameters are compared with prestored physiological index parameters, which are stored in a database, which is communicatively coupled to the processing system and define for each physiological parameter an optimal physiological range and at least one higher physiological range and at least one lower physiological range. The physiological index parameters are compiled from health guidelines from several international societies defining ideal and normal values for specific physiological parameters (such as recommendations from the European Society of Hypertension and the World Health Organization). In a preferred embodiment, the physiological index parameters are classified in up to five non-pathological subgroups around an optimal physiological range. For some physiological parameters (e.g. blood pressure), there is an optimal range and at least one higher range and one lower physiological range. For other physiological parameters (e.g. vascular age index), there is an optimal range and further physiological (higher) ranges, since the optimal value is as low as possible. The processing system is adapted to determine the deviation of the physiological parameter, that is determined, from the optimal physiological range and stratification of the user into the specific subgroup depending on the individual deviation from the optimal physiological range. Due to the stratification in up to five non-pathological subgroups, a more specific evaluation of the health status (such as cardiovascular health status) of the user subpopulations is achieved, with more parameters than evaluated in the state of the art.

A second database contains a list of nutrients, nutraceuticals, advanced food ingredients and single nutritional components specifically selected via scientific and clinical studies to have a specific positive/normalizing effect on said deviation(s) of physiological parameters from the optimal physiological range. Within this database it is specified, which nutrients are able to specifically influence (increase or decrease) the physiological parameter to reach the optimal physiological range as defined in the database with the prestored physiological index parameters. The nutrient database is based on scientific publications, showing specific effects for single nutrients or nutraceuticals with respect to specific physiological parameters. The processing system is adapted to search for scientific data for single nutrients or nutraceuticals within the database and provide a nutritional suggestion based on the individual deviations from the prestored physiological index parameters.

A third database containing general lifestyles, fitness and wellness information (recommendation) for comparing the deviation with the recommendations which are able to influence (increase or decrease) the physiological parameters. The processing system is adapted to provide a suggestion, which lifestyle, fitness or wellness information is suitable to influence (increase or decrease) the physiological parameter to reach the optimal physiological range as defined in the database with the prestored physiological index parameters.

Output means are adapted to output the calculated physiological parameters and the deviation from the prestored physiological index parameters and a nutritional suggestion for the user.

A supplementary visualization tool, such as a smartphone application is capable to run on different smartphones or personal computers. The system can further be complemented with a web-portal for further communication possibilities with the user and for the application/insertion-request of new supplements/functional food ingredients from the various suppliers. The visualization tool and the connected web portal provide detailed insights into the personal health status of the user and provides support for individually defined health or fitness targets of the user. Moreover, it contains personalized recommendations for nutrition for the user.

In an specific embodiment of the present invention, the processing system employs artificial intelligence (A.I.), which is capable to determine and stratify/classify the different physiological subgroups of the users (from the real measured data and related user's information) and generate the corresponding personalized new baseline of physiological parameters for such subgroup in the nutrient database ensuring a personalized selection of supplements and lifestyle recommendations from the nutrient database and the lifestyle database. In addition, the processing systems maintains updated both the nutrient and the lifestyle database via two distinct data-mining algorithms. The first data-mining algorithm related to the nutrient database is connected to scientific publications of private providers and public databases to extract dose-specific effects from new nutrients having a normalizing effect on specific physiological parameters to reach the optimal physiological range as defined in the database with the prestored physiological index parameters. The second data-mining algorithm is connected to the internet to extract new and supplementary lifestyle recommendations to be inserted into the lifestyle recommendation database. The final validation and subsequent insertion of the newly extracted information/recommendation into the related databases (nutrient database and lifestyle database), however, will be performed by human intelligence.

In another specific embodiment, the user generates specific feedback after nutritional suggestion and intake of the suggested nutrient. In a specific embodiment, the user feedback is entered via the visualization application or the web portal. Therefore, the processing system is configured evaluate the feedback of the user, if the suggested nutritional modification or lifestyle recommendation leads to an improvement of the physiological parameters. The processing system is configured to modify the nutritional suggestions and lifestyle recommendation based on the feedback of the user, which allows a more specific health assessment and a personalized recommendation for the user.

In further preferred embodiments, the described health monitoring system can be complemented with a series of connected devices or data entry points, which consider supplementary personal data for more accurate personalized nutrition suggestions.

These data can be derived but not limited to
a) Biomarker data, like blood glucose, lipid and cholesterol data, specific cytokines/inflammatory markers, hydration, etc.
b) DNA, RNA & Metabolomic data
c) Microbiome Analysis
d) Diet trackers and food analysis
e) Other devices, like balance, home devices (e.g. temperature and humidity control unit), voice control unit (e.g. Alexa), etc.

In an advantageous configuration the processing system is inked to an online marketing platform configured to visualize improvements and to directly order nutritional or nutraceutical products according to the suggestion provided.

In a further advantageous configuration, the processing system is linked to a mobile application configured to visualize improvements and to directly order nutritional or nutraceutical products according to the suggestion provided. The mobile application may also be configured to allow data input from various applications related to different health aspects, such as applications connected to a weight or applications relating to food tracking and determination of calorie consumption.

The system according to the present invention further also includes the possibility for the user to give feedback and enlarge the personalization level by integrating data from connected devices or analysis providers (e.g. DNA and Biomarker analysis).

It is further preferred that the user can share the physiological parameters, deviations from the prestored index parameters and improvements of physiological parameters with different partners of the Health Monitoring system, such as insurance companies, bonus-partners, trainers, practitioners, etc. The mobile application can also be coupled to different online platforms related to social media networks.

A further aspect of the present invention is a method for monitoring physiological parameters of a user comprising:
receiving input from at least one sensor and an interface of a human body health monitoring device of the user;
calculate one or more physiological parameters based on the primary physiological signals and based on individual parameters of the user,
comparing the calculated physiological parameters with prestored physiological index parameters, and determining a specific deviation between the calculated physiological parameters to the prestored physiological index parameters,
comparing the specific deviation(s) with a database containing nutrients, nutraceuticals, advanced food ingredients and single nutritional components specifically selected via scientific and clinical studies to have a specific positive/normalizing effect on said deviation(s),
providing a nutritional suggestion to the user for the normalization of the physiological parameters based on the comparison of the specific deviation(s) with the nutritional database; and
outputting the calculated physiological parameters, the deviation from the prestored index parameters and the nutritional suggestion.

In one embodiment of the present invention, the human body health monitoring device is a wrist-worn device for determining one or more of the following parameters:
the vascular age index Aglx,
the pulse wave velocity PVW,
blood pressure BP_{dia} and BP_{sys},
augmentation index AIx,

wherein the device comprises
two PPG sensors, with a distance of 5 cm or less, facing the dorsal part of the arm,
wherein the PPG sensor comprises at least one green light source and comprises a sampling frequency of preferably 512 Hz.

In a preferred embodiment, the device further comprises signal processing means adapted to calculate one or more of the following:
the vascular age index AgIx using linear regression based on the characteristic points a, b, c, d, and e, age (p_{age}), body height (p_{height}) and median heart rate of the subject,
the pulse wave velocity PWV using linear regression based on the time difference between the two PPG pulses (PTT), age (p_{age}), body height (p_{height}) and median heart rate estimation of the subject,
blood pressure BP_{dia} and BP_{sys} using linear regression based on time difference between the two PPG pulses (PTT) and median heart rate and
optionally the augmentation index AIx, based on the systolic A_{sys} and diastolic A_{dia} peak amplitudes normalized to 75 heartbeats (AIx@75) and using a linear regression based on the normalized augmentation index AIx,

The wrist-worn device can be a fitness tracker or a smartwatch.

### Embodiments of the Present Invention

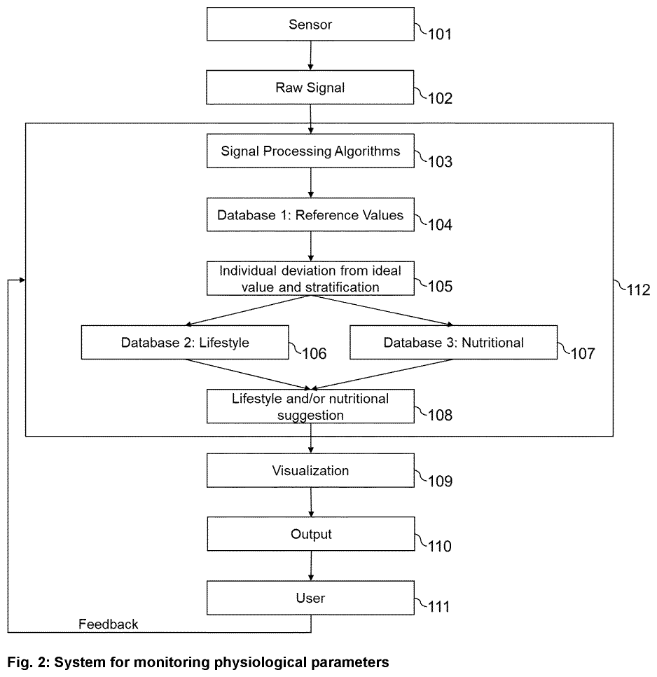

Embodiments of the present invention are displayed in FIGS. 2 to 6, wherein the reference numerals represent:

| | |
|---|---|
| 101 | One or more sensors able to measure at least cardiovascular |
| | parameters. |
| 102 | Raw signals measured by 101 (primary physiological signals) |
| 103 | Algorithms capable to extract the intended physiological |
| | parameters from 102 |
| 104 | Database containing reference values from national and/or |
| | international guidelines for physiological parameters |
| 105 | Based on physiological parameters from 103 and reference |
| | values in 104 individual deviation from ideal value is |
| | determined |
| 106 | Database containing information on lifestyles influencing each |
| | physiological parameter |
| 107 | Database containing information on nutrition and nutritional |
| | supplements influencing each physiological parameter |
| 108 | Individual suggestions based on 106 and 107 and 105 |
| 109 | Visualization of lifestyle and/or nutritional suggestion |
| 110 | Output of the lifestyle and/or nutritional suggestion |
| 111 | Feedback of the user to the processing system |
| 112 | Processing system |
| 113 | Control unit |
| 200 | System for determining cardiovascular parameters |
| 201 | PPG sensor |
| 212 | Processing system |
| 213 | Memory |
| 214 | Comparison with prestored data |
| 215 | User interface |

FIG. 2 shows a system for monitoring physiological parameters according to the present invention. The system includes one or more sensors, which are configured to measure one or more physiological parameters. At least one of these sensors is included within a human body health monitoring device.

The system further comprises a processing system communicatively coupled to the sensor and adapted to calculate one or more physiological parameters based on the primary physiological signals and based on individual parameters of the user. The raw signals (primary physiological signals) 102 are directly measured and then further processed using signal processing algorithms 103.

The signal processing algorithms are configured in a way that they are capable to extract the desired parameters from the raw signals 102. The system further comprises several databases. Database 1 contains reference values from national and/or international guidelines (prestored physiological index parameters) for the physiological parameters which are to be determined 104. The calculated physiological parameters are compared with prestored physiological index parameters, which are stored in a database, which is communicatively coupled to the processing system and define for each physiological parameter an optimal physiological range and at least one higher physiological range and at least one lower physiological range. The physiological index parameters are compiled from health guidelines from several international societies defining ideal and normal values for specific physiological parameters (such as recommendations from the European Society of Hypertension and the World Health Organization). The physiological index parameters are classified in up to five non-pathological subgroups around an optimal physiological range. The processor 112 then compares the calculated physiological parameters with prestored physiological index parameters in Database 1 and determines the specific deviation between the calculated physiological parameters and the prestored physiological index parameters 105.

The system further comprises a database containing nutrients, nutraceuticals, advanced food ingredients and single nutritional components specifically selected via scientific and clinical studies to have a specific positive/normalizing effect on the physiological parameters (Database 3) 107. Within this database it is specified, which nutrients are able to specifically influence (increase or decrease) the physiological parameter to reach the optimal physiological range as defined in the database with the prestored physiological index parameters. The nutrient database is based on scientific publications, showing specific effects for single nutrients or nutraceuticals with respect to specific physiological parameters. The processing system is adapted to search for scientific data for single nutrients or nutraceuticals within the database and provide a nutritional suggestion based on the individual deviations from the prestored physiological index parameters 108.

Another database (Database 2) 106 contains general lifestyles, fitness and wellness information (recommendation) for comparing the deviation with the recommendations which are able to influence (increase or decrease) the physiological parameters. The processing system is adapted to provide a suggestion, which lifestyle, fitness or wellness information is suitable to influence (increase or decrease) the physiological parameter to reach the optimal physiological range as defined in the database with the prestored physiological index parameters. The processing system is adapted to further provide a lifestyle suggestion based on the individual deviations from the prestored physiological index parameters 108.

Output means 110 are adapted to output the calculated physiological parameters and the deviation from the prestored physiological index parameters and a nutritional suggestion for the user. The individual suggestions 108 are visualized for the user in a mobile application and/or in a web portal 109. The user 111 can provide feedback 111 to the system, which ensures validation of the suggestions and normalization of the physiological parameters based on the comparison of the specific deviation with the nutritional database.

The analysis of the cardiovascular parameter estimation has shown that there are multiple cardiovascular parameters that can be estimated with reasonable deviation from the reference using PPG signals. To conclude, the simple and low-cost PPG signal contains useful information about a person's cardiovascular health that lay far beyond the pulse rate, which is currently the most common extracted feature. The novel algorithms can estimate cardiovascular parameters with only a slight deviation from the reference values even in case of two PPG sensors located at the wrist. This offers for the first time the possibility to include two PPG sensors within one wrist-worn device to provide a detailed analysis of the cardiovascular conditions of a subject. The two PPG sensors can be included into a fitness tracker or a smartwatch for permanent monitoring of those cardiovascular parameters.

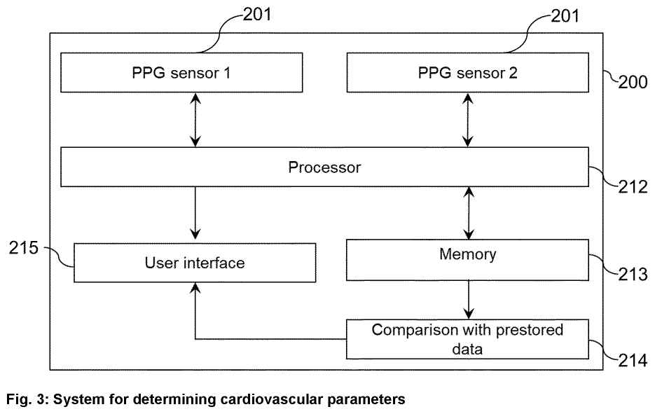

FIG. 3 exemplarily shows a system 200 for determining cardiovascular parameters, such as vascular age index AgIx, blood pressure BP_{dia} and BP_{sys}, pulse wave velocity PWV, augmentation index Alx and heart rate variability HRV. The system 200 can be implemented in a wrist-worn human body health monitoring device, such as a fitness tracker or a smartwatch and includes two PPG sensors 201, a processor 212, a memory 213, comparison with prestored data 214 and a user interface 215. The database 213 contains reference data for all cardiovascular parameters and may be derived from physiological data obtained from different organizations databases and obtained from measured data of the system 200. In another embodiment, a database can be externally coupled to the system through wired or wireless connectivity.

The two PPG sensors 201 are configured to illuminate skin of a user and measure two PPG signals based on the illumination absorption by the skin. The PPG sensors 201 may include, for example, at least one periodic light source (e.g., light-emitting diode (LED), or any other periodic light source related thereof), and a photo detector configured to receive the periodic light emitted by the at least one periodic light source reflected from the user's skin. In a preferred embodiment, the PPG sensor comprises at least one green light source and comprises a sampling frequency of preferably 512 Hz.

The two PPG sensors 201 can be coupled to the processor 212. In another embodiment, the PPG sensors 101 may be included in a housing with the processor 212 and other circuit/hardware elements. It is preferred, when both PPG sensors 201 are included in a housing and are positioned with a distance of 5 cm or less, facing the dorsal part of the arm.

The processor 212 (for example, a hardware unit, an apparatus, a Central Processing Unit (CPU), a Graphics Processing Unit (GPU)) can be configured to receive and process the periodic light received from the PPG sensors 201. The processing includes pre-processing of the data at first instance as discussed before and estimation of the cardiovascular parameters with help of the algorithms according to the present invention. The estimated cardiovascular parameters are then compared with prestored data 214 and processed to the user interface 215 to be displayed for the user. The user can further provide feedback to the estimated parameters.

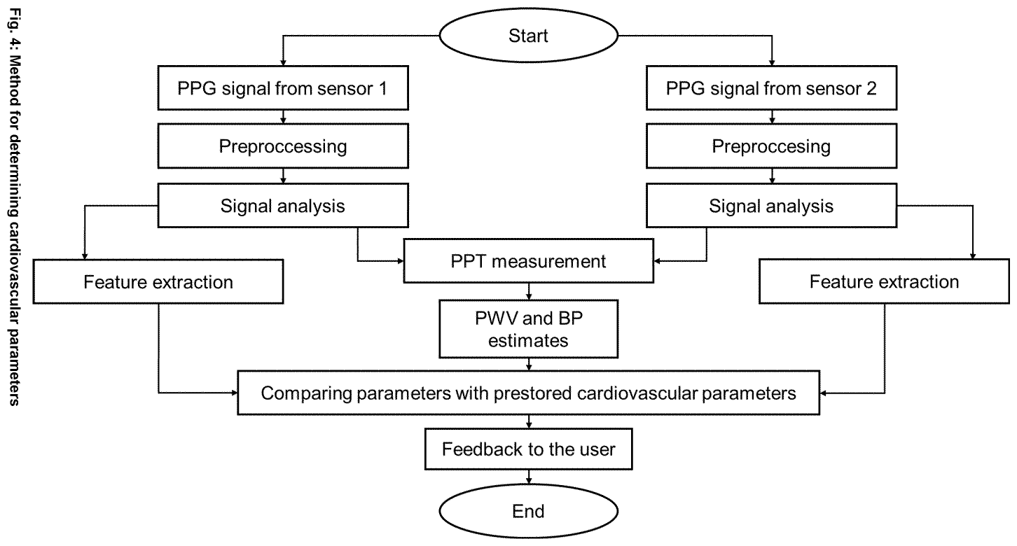

FIG. 4 is a flow diagram illustrating a method for estimating one or more cardiovascular parameters in a subject, according to an exemplary embodiment based on two PPG signals from two separate PPG sensors. Referring to FIG. 4, in operation, the electronic device illuminates skin of a user and measures the PPG signal from two PPG sensors based on the illumination absorption by the skin. For example, in the electronic device, as illustrated in FIG. 3, the two PPG sensors 201 are configured to illuminate the skin of the user and measure the PPG signal based on am illumination absorption by the skin.

In operation, the system 200 extracts a plurality of parameters from both PPG signals, after preprocessing of the signal, including the PPG features, the HRV features, the APG features and the pulse transit time (PTT). Based on the two PPG signal analysis, the cardiovascular parameters can be estimated as described above. The system 200 estimates the cardiovascular parameters, in this case PWV and BP based on the extracted plurality of parameters. The estimated parameters are compared with prestored cardiovascular parameters 214. The result is displayed within the user interface 215 giving feedback to the user.

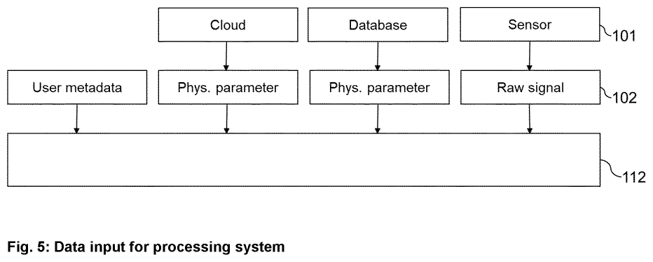

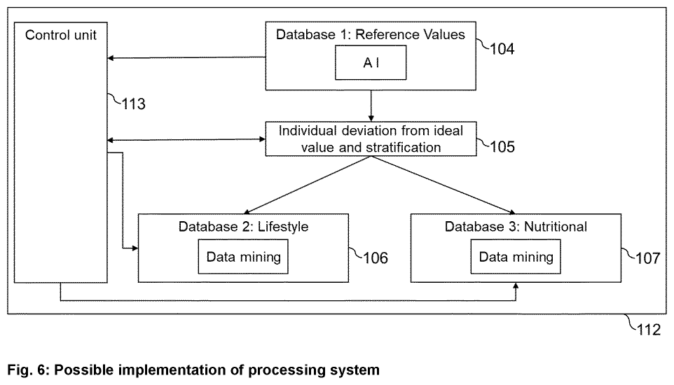

FIG. 5 shows different sources for data input into the processing system 112, especially into the control unit 113 (as shown in FIG. 6). Primary sensor data are directly provided by a sensor 101, such as a PPG sensor as raw signals 102, such as PPG signals into the processing system for further processing of the raw data into physiological parameters, such as blood pressure. For the determination of specific physiological parameters different metadata of the user are additionally required. Therefore, user metadata are entered into the processing system 112, especially age, height, weight, gender, fitness level, anamnesis data. These data are also required to allow specific personalized suggestions, which are in line with the behavior and the overall health status of the user. Further information on activities, drinking and eating behavior, sleep times may also be entered by the user into the processing system 112. Further data entry might be related to physiological parameters of the user, which are externally stored, in a data cloud for example. These data can be derived from different connected devices or mobile applications, which are connected with such devices or applications, which are manually updated by the user. Physiological parameters may also be entered from a database, which is connected to such devices or applications.

Further data input can be
a) Biomarker data, like blood glucose, lipid and cholesterol data, specific cytokines/inflammatory markers, hydration, etc.
b) DNA, RNA & Metabolomic data
c) Data from microbiome Analysis
d) Data from diet trackers and food analysis
e) Data from other devices, like balance, home devices (e.g. temperature and humidity control unit).

FIG. 6 display one possible implementation of the processing system 112, wherein the processing system 112 comprises a control unit 113, which communicates between the different databases. In this implementation of the present invention, the processing system employs artificial intelligence (A.I.) within the reference values database, which is capable to determine and stratify/classify the different physiological subgroups of the users (from the real measured data and related user's information) and generate the corresponding personalized new baseline of physiological parameters for such subgroup. By comparing the measured physiological value with the reference values database 104, the individual deviation from the ideal values 105 is determined. This ensures a personalized selection of supplements and lifestyle recommendations from the nutrient database 107 and the lifestyle database 106. In addition, the processing systems maintains updated both the nutrient and the lifestyle database via two distinct data-mining algorithms. The first data-mining algorithm related to the nutrient database is connected to scientific publications of private providers and public databases to extract dose-specific effects from new nutrients having a normalizing effect on specific physiological parameters to reach the optimal physiological range as defined in the database with the prestored physiological index parameters. The second data-mining algorithm is connected to the internet to extract new and supplementary lifestyle recommendations to be inserted into the lifestyle recommendation database. The final validation and subsequent insertion of the newly extracted information/recommendation into the related databases (nutrient database and lifestyle database), however, will be performed by human intelligence.

With the help of the system and the method for estimating one or more cardiovascular parameters, the user can continuously monitor and evaluate physiological parameters, such as cardiovascular parameters. Based on the advanced algorithms including specific anatomical data, the evaluation of several cardiovascular parameters is achieved. The evaluation of supplementary parameters, such as blood flow, blood pressure, arterial stiffness, vessel elasticity, vascular age allows a comprehensive general health assessment. This individual cardiovascular health assessment reduces the risk of misinterpretation and leads to a more precise health assessment for the user.

Parameters for Health Assessment

Primary parameters, which are considered for the health assessment are selected from
Basic user descriptors: age, weight, height
Further user descriptors: smoking, allergies
Sleep quality, duration
Calorie burn
Activity (steps, distance)
Hearth rate variability
Blood pressure
Pulse wave velocity
Stress
Blood oxygen saturation

Further primary parameters are selected from
VO₂ₘₐₓ
Light exposure
Recovery index
Skin temperature
Skin blood perfusion
Skin hydration
Performance index
Calorie intake (food registration)
Body composition (water, fat, muscle)
BMI
Heart rate
Glucose level

Secondary parameters, which are considered for the overall health assessment are selected from
Stress
Sleep index
Basal metabolic rate
Recommended calorie intake

Further secondary parameters are selected form
Hydratation level
Temperature variation
Body temperature
Vitamin D warning
Augmentation Index
Inflammation/Infection
Hydratation warning
Energy expenditure

Additionally, environmental parameters can be considered for an optimal health assessment:
Light exposure (external)
Atmospheric temperature
Humidity
Atmospheric pressure
Altitude
Pollution

Moreover, results from specific analysis can be considered for further assessment:
DNA analysis
Blood work
Gut-microbiome analysis

### WORKING EXAMPLE

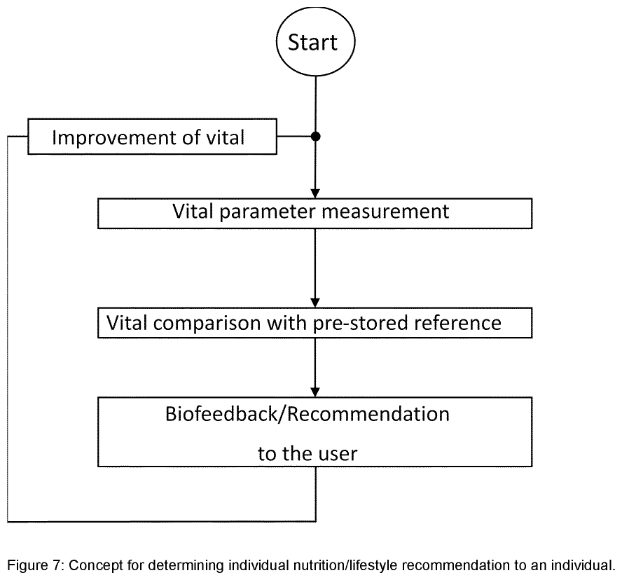

Nutrition and lifestyle behaviors have a significant influence on the wellbeing of on individual. This wellbeing can be verified by estimating the individual vital parameters. An exemplary but not limiting list of such vital parameters are cardiovascular parameters (heart rate, blood pressure, pulse wave velocity), stress level, and sleep indicators like sleep quality and latency. Exemplary but not limiting correlations between nutrition and their influence on such vital parameters are shown in Table 1. The following concept explains the determination of individual nutrition/lifestyle recommendations to an individual (FIG. 7).

TABLE 1
Overview on integrated vital parameter with
nutrition recommendation for an improvement.

| Vital parameter | Nutrition Recommendation |
|---|---|
| Sleep quality/latency | Vitamins D, Amino acids, Food supplements |
| | based on magnesium or zinc |
| Stress | Omega-3 fatty acids |
| Heart Rate | Omega-3 fatty acids |
| Blood pressure | Anthocyanins, Omega-3 fatty acids |
| Pulse wave velocity | Anthocyanins, Omega-3 fatty acids |

For an individual recommendation, a measurement of vital parameters of the individual must be conducted. This can be done in a continuous manner (continuous session) over a certain time period. An example of such a continuous session is a photoplethysmography (PPG) based measurement (with PPG sensors integrated in a fitness tracker) of a population. The obtained PPG signal are then used to calculate specific cardiovascular physiological parameters, via the algorithm according to the specific embodiments of the present invention.

Pilot Study (Continuous PPG Measurement to Monitor Cardiovascular Parameters)

A pilot study was conducted to analyze the functionality of the present invention. 22 healthy individuals (age: 29-59 years, gender: 82% male, 18% female) continuously measured their physiological parameters with a human body health monitoring device (fitness tracker), comprising two PPG sensors. In general, per day, two PPG-measurements for each user were performed and thereby primary physiological signals were obtained for each individual. The physiological parameters of the individuals were collected for 14 days, during which over 1800 cardiovascular parameters were calculated in total and 60 personal suggestions were given, based on deviations of calculated cardiovascular parameters from reference values. The cardiovascular parameters and the suggestions were displayed to each individual via a mobile application on a mobile device.

Based on the measured PPG signals and the specific parameters of the user: age, gender, height and weight of the user, the physiological parameters vascular age index (AgIx), pulse wave velocity (PWV), blood pressure (BP_{dia} and BP_{sys}) and were calculated using the algorithms:
a) vascular age index AgIx:
   AgIx=d₀+d₁ [symbol] +d₂p_{age}+d₃p_{height}+d₄ [symbol] , wherein [symbol] is estimated based on characteristic points a, b, c, d, and e:

$$= 45.4 * \frac{b - c - d - e}{a} + 65.9 ;$$

b) pulse wave velocity PWV:

PWV=g₀+g₁ [symbol] +g₂p_{age}+g₃p_{height}+g₄ [symbol] ;
c) blood pressure BP_{dia} and BP_{sys}:

BP_{dia}=l_{0d}+l_{1d} [symbol] +l_{2d} [symbol] +l_{3d}CTₚ+l_{4d}SIₚ+l_{5d}PAₚ

BP_{sys}=k₀ₛ+k₁ₛ [symbol] +k₂ₛ [symbol] ;
wherein, p_{age} is the age and p_{height} is the body height of the subject, median (HR) is the median heart rate, PTT is the time difference between the PPG pulses, A_{sys} and A_{dia} are magnitudes of the systolic and diastolic peak, respectively, CT is the Crest Time, ST is the Stiffness Index and PA is the Pulse Area of the PPG signal, d₀ to d₄, g₀ to g₄, l_{0d} to l_{kd}, k₀ₛ to k₂ₛ, and b₀ to b₁ represent the coefficients of the respective linear regression equation.

The median heart rate was determined from the PPG signal and the Heart Rate Variability (HRV) was determined based on the median heart rate and the Root Mean Square of Successive Difference between normal heartbeats (RMSSD). The RMSSD was obtained by first calculating each successive time difference between heartbeats and then, each of the values is squared and the result is averaged before the square root of the total.

The calculated values for the physiological parameters were compared with pre-stored reference values (prestored physiological index parameters) relating to age, gender, height and weight of the user. Those reference values were summarized from the European Society of Hypertension (ESH) and of the European Society of Cardiology (ESC) and Bel Marra Health, and the deviation between the calculated physiological parameter and physiological index parameter was determined for each calculation.

A database was prepared, based on scientific publications indicating beneficial effects of single nutritional elements on said physiological parameters.

When a deviation from the reference values was determined, a nutritional suggestion was displayed (biofeedback/recommendation to the user), in order to achieve an improvement of said physiological parameter and overall cardiovascular health of the user.

The nutritional suggestion was outputted in a mobile application on a mobile device (output means). The user could then also provide feedback on health status via the mobile application run on a mobile phone.

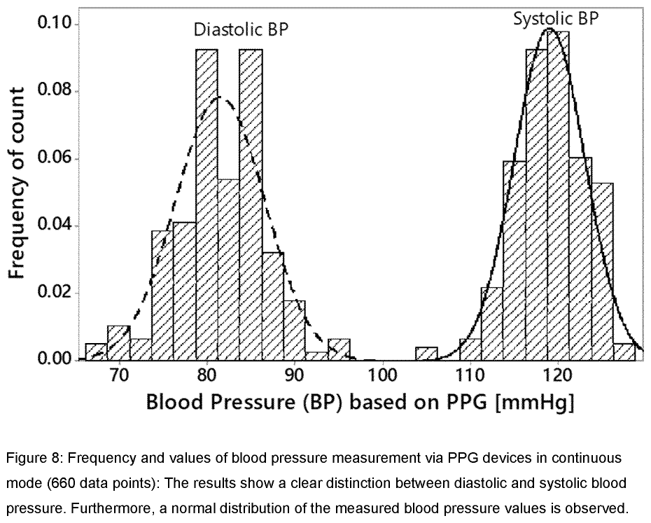

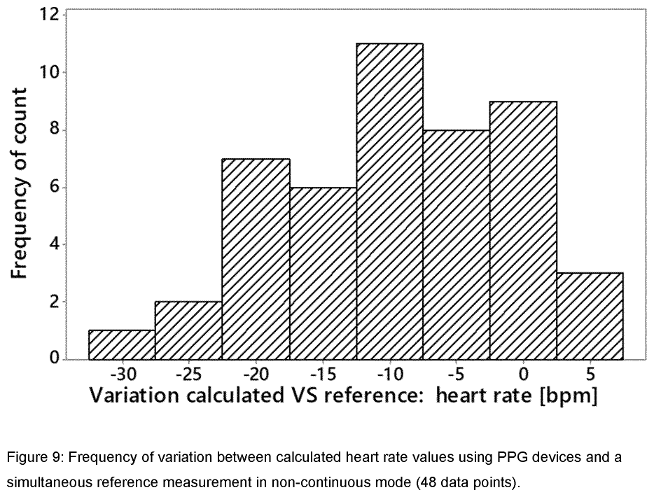

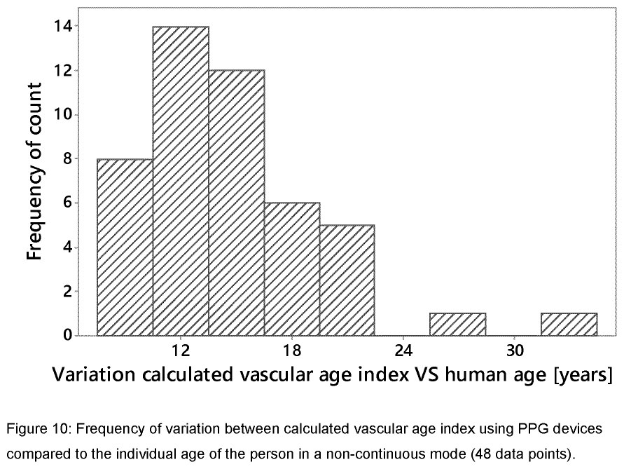

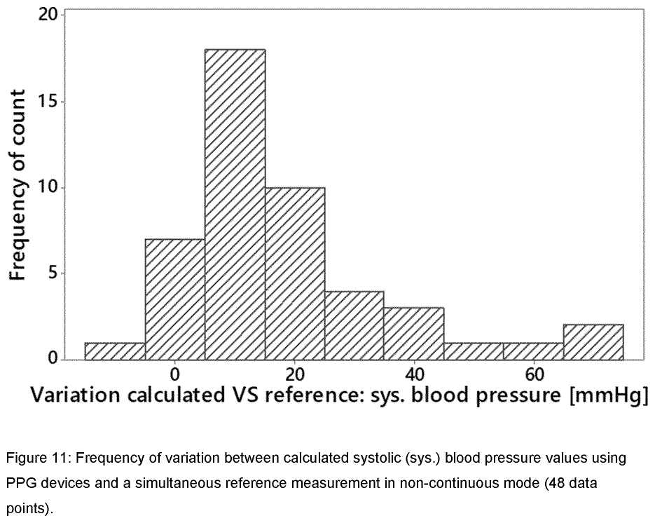

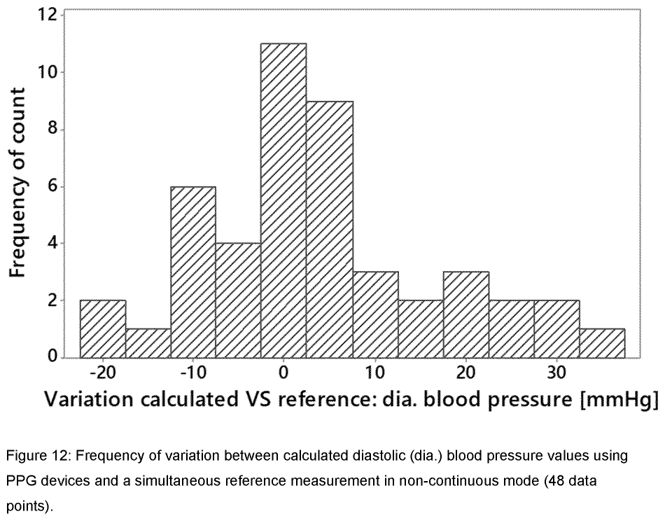

One example of such a continuous session is continuous blood pressure measurement, with a total of 660 data points, which is displayed in FIG. 8. The figure shows the calculated blood pressures of a population (22 individuals) and the frequency of count of each blood pressure value inside the population. The results show a clear distinction between a diastolic and systolic blood pressure of the population. Furthermore, a normal distribution in the counts of blood pressure values can be observed (visibly shown by Gaussian function). In a control session, the used technology, was also compared to a simultaneous reference technology (sphygmomanometer). As an example of a control session, PPG measurements and vital calculations via the mentioned algorithm are compared to a simultaneous reference technology via a sphygmomanometer. An example of such a control session, with 48 data points, can be found in FIG. 9 (heart rate), FIG. 10 (vascular age index), FIG. 11 (systolic blood pressure) and FIG. 12 (diastolic blood pressure). The figures are showing the frequency of variations between calculated values using PPG devices and a simultaneous reference measurement.

After calculation of the physiological parameters, a comparison to a pre-stored reference value was conducted. Examples of such a comparison for four individuals (named A, B, C, D) in a population is summarized in table 2 and table 3. After comparison of the calculated physiological parameter with prestored physiological index parameters, the measured blood pressure (shown in table 2) and/or heart rate (shown in table 3) of each individual was classified in one of five prevention classes. Such prevention class can be for example “optimal”, “slightly higher than optimal” or “higher than optimal”. For each classified prevention class, a specific recommendation (Rec.) was outputted (summarized in table 4), e.g. user A had optimal values for blood pressure and the recommendation “0” was outputted via the mobile application, which means that no change of behavior is required.

TABLE 2
Individual recommendations (Rec.) for blood pressure
improvement bases on continuous PPG measurement;
with classification in prevention class.

| | Blood Pressure | | | |
|---|---|---|---|---|
| | (Average ± Deviation) | | | |
| | [mmHg] | | Range | |
| Example | (Systolic) | (Diastolic) | [Systolic/Diastolic] * | Rec. |
| A | 116.92 ± 0.93 | 82.25 ± 1.79 | Optimal/optimal | 0 |
| B | 122.14 ± 1.76 | 88.43 ± 1.29 | Optimal/slightly | 1 |
| | | | higher than optimal | |
| C | 123.75 ± 0.69 | 93.25 ± 2.19 | Optimal/higher | 2 |
| | | | than optimal | |

* Blood pressure prevention class according to the European Society of Hypertension (ESH) and of the European Society of Cardiology (ESC)

TABLE 3
Individual recommendations (Rec.) for heart rate
improvement bases on continuous PPG measurement;
with classification in prevention class.

| | | Heart Rate | | |
|---|---|---|---|---|
| | | (Average ± Deviation) | | |
| Example | Age/Gender | [Beats per minute] | Range ^{#} | Rec. |
| A | 59/male | 61.65 ± 6.49 | Optimal | 0 |
| B | 32/female | 80.73 ± 1.76 | Higher than | 3 |
| | | | optimal | |
| D | 32/male | 70.92 ± 1.6 | Slightly higher | 3 |
| | | | than optimal | |

^{#} Heart rate prevention class according to Bel Marra Health considering age and gender influence

According to the prevention class for each physiological parameter, an individual recommendation for each user was generated and outputted via the mobile application on a mobile phone. As an example, the four individual recommendations from tables 2 and 3 are summarized in table 4. In case of the optimal values for physiological parameters, a biofeedback can include the information that the nutrition/lifestyle behavior is optimal, and no modification is needed “recommendation: 0” (table 4). In the case of a non-optimal physiological parameter (e.g. a blood pressure and heart rate higher than optimal for user B), biofeedback can give a recommendation on nutrition/lifestyle variation to the individual. In this example, information is given on lowering blood pressure and/or heart rate by a quantitative daily intake of specific substances “recommendation: 1+3” (table 4). Those recommendations are based on published literature (table 4). The influence of such nutrition/lifestyle variation on the improvement of the vital parameters can be measurable through continuous measurement.

TABLE 4
Individual recommendations to lower blood pressure and heart rate values,
with quantitative daily intake information, and references to literature.

| Recommendation | Daily intake | Reference-DOI | Reference-Article |
|---|---|---|---|
| 0 | No change of behavior needed | | |
| 1 | 1.5 g | 10.1016/j.jpeds.2010.04.001 | 2010, The Journal of pediatrics, |
| | Fish oil | | Vol. 157, No. 3, pp. 395-400 |
| 2 | 300 mg | 10.1177/2156587213482942 | 2013, Journal of Evidence-Based |
| | Anthocyanin | | Complementary & Alternative |
| | | | Medicine, 18, 4, 237-242 |
| 3 | 0.85-3.4 g | 10.1016/j.atherosclerosis.2013.10.014 | 2014, Atherosclerosis, 232, 1, 10- |
| | Omega-3 fatty acids | | 16 |

1. A system for monitoring physiological parameters of a user, the system comprising:
a human body health monitoring device comprising a sensor adapted to obtain primary physiological signals of the user;
a processing system communicatively coupled to the sensor adapted to
calculate one or more physiological parameters based on the primary physiological signals and based on individual parameters of the user,
compare the calculated physiological parameters with prestored physiological index parameters, and determine a specific deviation between the calculated physiological parameters to the prestored physiological index parameters,
compare the specific deviation(s) with a database containing nutrients, nutraceuticals, advanced food ingredients and single nutritional components specifically selected via scientific and clinical studies to have a specific positive/normalizing effect on said physiological parameters,
provide a nutritional suggestion to the user for the normalization of the physiological parameters based on the comparison of the specific deviation(s) with the nutritional database; and
output means adapted to output the calculated physiological parameters, the deviation from the prestored index parameters and the nutritional suggestion.

2. The system of claim 1, wherein the physiological parameters calculated are cardiovascular health parameters, cognitive health parameters, gut health parameters, metabolic parameters, body mass and body efficiency parameters, stress and sleep parameters or inflammatory parameters or a combination.

3. The system of claim 1, wherein the prestored physiological index parameters are stored in a database, which is communicatively coupled to the processing system and define for each physiological parameter an optimal physiological range and at least one higher physiological range and at least one lower physiological range.

4. The system of claim 1, wherein the sensor is a photoplethysmographic (PPG) sensor and the physiological parameters calculated are cardiovascular health parameters selected from the group consisting of vascular age index AgIx_{PPG}, blood pressure BP_{dia} and BP_{sys}, pulse wave velocity PWV, augmentation index AIx_{PPG}, and heart rate variability HRV.

5. The system of claim 1, further comprising at least one selected from the group consisting of bioimpedance sensor, pulse oximeter, capacitive sensor, temperature sensor, ultraviolet (UV) sensor, ambient light sensor, 3 axis accelerometer, altimeter, barometer, compass, gyroscope, magnetometer, gesture technology, global positioning system (GPS), and long term evolution (LTE).

6. The system of claim 1, wherein the physiological parameters, the primary physiological signals, individual parameters of the subject and nutritional suggestion to the user for the normalization of the physiological parameters are collected to establish a database for comparison and detection of deviations.

7. The system of claim 1, wherein the system is configured to determine one or more of the following cardiovascular parameters of the user, the user having an age and a body height with the following steps:
determining the age (p_{age}) and body height (p_{height}) of the user,
measuring at least two photoplethysmographic (PPG) signals with at least two PPG sensors at two different positions at the subject,
separating the PPG signal into PPG pulses, whereby the start point and the end point of the pulse corresponds the systolic foot of the PPG signal,
determining the heart rate of the user (p_{HR}) and calculating the median heart rate,
determining the systolic A_{sys} and diastolic A_{dia} peak amplitudes and their times tₛ and t_{d},
calculating the second derivative of the PPG pulse, and determining the characteristic points a, b, c, d, and e from the second derivative of the PPG pulse, wherein
a and e are the first and second most prominent maxima in the second derivative, respectively,
c is the most prominent peak between the points a and e,
b is the most prominent minimum in the second derivative and,
d is the most prominent minimum between points c and e,
determining:
a) the vascular age index AgIx using linear regression based on the characteristic points a, b, c, d, and e, age (p_{age}), body height (p_{height}) and median heart rate of the user,
b) the pulse wave velocity PWV using linear regression based on the time difference between the two PPG pulses (PTT), age (p_{age}), body height (p_{height}) and median heart rate estimation of the user,
c) blood pressure BP_{dia} and BP_{sys} using linear regression based on time difference between the two PPG pulses (PTT) and median heart rate and
d) optionally the augmentation index AIx, based on the systolic A_{sys} and diastolic A_{dia} peak amplitudes normalized to 75 heartbeats (AIx@75) and using a linear regression based on the normalized augmentation index AIx.

8. The system of claim 1, further comprising the determination of Crest Time (CT), Stiffness Index (SI) and Pulse Area (PA) of the PPG signal and wherein the cardiovascular parameters are estimated with the following equations:
a) vascular age index AgIx:
AgIx=d₀+d₁ [symbol] +d₂p_{age}+d₃p_{height}+d₄ [symbol] , wherein [symbol] is estimated based on characteristic points a, b, c, d, and e:
$$= 45.4 * \frac{b - c - d - e}{a} + 65.9 ;$$
b) pulse wave velocity PWV:
PWV=g₀+g₁ [symbol] +g₂p_{age}+g₃p_{height}+g₄ [symbol] ;
c) blood pressure BPdia and BPsys:
BP_{dia}=l_{0d}+l_{1d} [symbol] +l_{2d} [symbol] +l_{3d}CTₚ+l_{4d}SIₚ+l_{5d}PAₚ
BP_{sys}=k₀ₛ+k₁ₛ [symbol] +k₂ₛ [symbol] ;
d) normalized augmentation index AIx@75: [symbol]
=(x−y)/y by the sum of two exponential, and
AIx@75=b₀+b₁ [symbol] , wherein AIx@75 is the augmentation index (AIx) normalized to 75 heartbeats;
wherein, p_{age} is the age and p_{height} is the body height of the subject, median (HR) is the median heart rate, PTT is the time difference between the PPG pulses, A_{sys} and A_{dia} are magnitudes of the systolic and diastolic peak, respectively, CT is the Crest Time, ST is the Stiffness Index and PA is the Pulse Area of the PPG signal, d₀ to d₄, g₀ to g₄, l_{0d} to l_{kd}, k₀ₛ to k₂ₛ, and b₀ to b₁ represent the coefficients of the respective linear regression equation.

9. The system of claim 7, wherein the characteristic points a, b, c, d, and e are automatically derived from the second derivative of the PPG pulse, wherein
a and e are the first and second most prominent maxima in the second derivative, respectively,
c is the most prominent peak between the points a and e,
b is the most prominent minimum in the second derivative and,
d is the most prominent minimum between points c and e.

10. The system of claim 7, wherein the systolic A_{sys} and diastolic A_{dia} peak amplitudes and their times tₛ and t_{d} are determined by one of the following methods:
modeling the PPG waveform as a sum of two pulse waves through exponential functions and applying nonlinear regression to fit the model to the PPG waveform and receive estimates of tₛ and t_{d} to find A_{sys} and A_{dia}, respectively, or
modeling the first wave with known position at the systolic peak A_{sys}, and subtracting its exponential model from the PPG signal and thereby yielding the remaining reflected wave, whose maximal value Â_{dia} and \hat{t}_{d} is the corresponding diastolic time index estimate.

11. The system of claim 1, further comprising an online marketing platform, wherein the processing system is linked to the online marketing platform configured to visualize improvements and to directly order nutritional or nutraceutical products according to the suggestion provided.

12. The system of claim 1, further comprising an application, wherein the processing system is linked to the application configured to visualize improvements and to directly order nutritional or nutraceutical products according to the suggestion provided.

13. A method for monitoring physiological parameters of a user, the method comprising:
receiving input from at least one sensor and an interface of a human body health monitoring device of the user;
calculate one or more physiological parameters based on the primary physiological signals and based on individual parameters of the user,
comparing the calculated physiological parameters with prestored physiological index parameters, and determining a specific deviation between the calculated physiological parameters to the prestored physiological index parameters,
comparing the specific deviation(s) with a database containing nutrients, nutraceuticals, advanced food ingredients and single nutritional components specifically selected via scientific and clinical studies to have a specific positive/normalizing effect on said physiological parameters,
providing a nutritional suggestion to the user for the normalization of the physiological parameters based on the comparison of the specific deviation(s) with the nutritional database; and
outputting the calculated physiological parameters and the nutritional suggestion.
